(19) United States
(12) Patent Application Publication
(10) Pub. No.: US 2003/0215783 A1
(43) Pub. Date: Nov. 20, 2003

(54) Genetic markers for semen viability

(76) Inventors: Lisa M. Thurston, London (GB); William V. Holt, London (GB); Paul Watson, London (GB); Alan J. Mileham, Walnut Creek, CA (US)

Correspondence Address: HUNTON & WILLIAMS, INTELLECTUAL PROPERTY DEPARTMENT, 1900 K STREET, N.W., SUITE 1200, WASHINGTON, DC, 20006-1109, US

(21) Appl. No.: 10/347,515

(22) Filed: Jan. 21, 2003

Related U.S. Application Data
(60) Provisional application No. 60/349,232, filed on Jan. 18, 2002.

(51) Int. Cl. A01N 1/02; C12Q 1/68

(52) U.S. Cl. 435/2; 435/6

(57) ABSTRACT

A method for identifying genetic markers for the viability of sperm following freezing, using genome scanning techniques is disclosed. In an embodiment of the invention, a method for identifying the presence or absence of genetic markers associated with viability of pig semen following cryopreservation comprises the steps of: obtaining nucleic acid from a pig, and identifying the presence or absence of one or more markers for semen viability. Preferably, the identification of markers involves amplified restriction fragment length polymorphism techniques.

The present invention relates to a method for identifying genetic markers for the viability of sperm following freezing, using genome scanning techniques.

Semen preservation by means of freezing (cryopreservation) is an integral tool in maintaining and manipulating valuable genetic resources. A long-term semen storage system is advantageous allowing movement of artificial insemination (AI) doses throughout the world without compromising semen viability. Long-term storage also enables specific health checks to be carried out on both the semen and individual males thus minimising the risk of the spread of disease through AI.

Global genetic selection pools can be created by using successfully cryopreserved semen for AI and this increases breeding potential, while being cost effective and reduces the spread of disease. However cryopreservation of boar spermatozoa is problematic. Not only is the spermatozoa highly sensitive to cryoinjury but a large sperm number and inseminate volume is required. Improvements in the understanding of stresses exerted on the spermatozoa during cryopreservation and an increased efficiency in freezing methods will be reflected in the choice of cryopreservation as a viable option for porcine AI.

Although semen cryopreservation has been applied successfully in several species, extensive variation in post-thaw semen quality exists between individuals (Reed HBC (1985) Current use of frozen boar semen. In: Deep freezing of boar semen, Johnson L A & Larsson K (eds). Uppsala: Swedish University of Agricultural Sciences). The Viability of the sperm may be determined using e.g. SYBR-14 and propidium (Garner D L & Johnson L A. (1995) Viability assessment of mammalian spermotozoa using SYBR-14 and Propidium. Iodide Biology of Reproduction 53:276-284; Holt C, Holt W V, Moore H D M, Reed H C B & Curnock R M. (1997) Objectively measured boar sperm motility parameters correlate with outcomes of on-farm inseminations: results of two fertility trials. Journal of Andrology 18: 312-323). Evidence indicates that consistent inter-individual variation in sperm viability following freezing is genetically determined. Although previous studies that have indicated that there is significant variation in post-thaw semen quality between boars following cryopreservation, it has not been investigated if the variation between boars that provide good or poor post-thaw semen quality has a genetic basis.

The identification of genetic differences between individuals linked to cryosurvival, offers a new approach with which to explore sperm cryobiology and is of direct relevance to the animal-breeding industry. Current advances in the understanding of the causes of variation at the genetic level will offer some explanation for the phenomenon of good and bad freezers and provide opportunities to improve the quality of cryopreserved semen through selective breeding.

The amplified restriction length polymorphism (“AFLP”) technique (Vos et al., 1995) visualises differences in DNA sequences between individuals and provides a means to identify DNA markers associated with desired traits. AFLP is an established molecular tool for quantifying genetic variation at the individual, population and species level among botanists (Tohme et al., 1996; Travis et al., 1996) and mycologists (Majer et al., 1996; Rosendahl & Taylor, 1997). However, the potential application of AFLP technology for animal research has only been recognised in a limited number of studies (Ajmone-Marsan et al., 1997). The implementation of AFLP markers to screen boars for semen ‘freezability’ requires that both the technique and the markers are reproducible. AFLP technology produces robust and reliable molecular markers through the high stringency PCR technique used for primer annealing (Matthes et al., 1998.) Alternative techniques include but are not limited to random amplified polymorphic DNA analysis (RAPD) and genome scanning with microsatellites or single nucelotide polymorphisims (SNPs).

In the first aspect the present invention provides a method for identifying the presence or absence of genetic markers associated with viability of pig semen following cryopreservation comprising of:

(a) Obtaining nucleic acid from a pig; and

(b) Identifying the presence or absence of one or more markers for semen viability.

The nucleic acid may be obtained from a pig prior to sexual maturity or from pig semen.

The nucleic acid is preferably DNA.

Preferably, the identification of markers involves amplified restriction fragment length polymorphism techniques.This method uses restriction enzymes to preferably produce fragments of 300-500 base pairs. For example EcoRI and TaqI can be used.

The fragments are preferably amplified using pairs of primers. In one embodiment one or more of the following pairs of primers can be used:

GTA GAC TGC GTA CCA ATT C AAG and GAT GAG TCC TGA CCG A AAC; or
GTA GAC TGC GTA CCA ATT C AAC and GAT GAG TCC TGA CCG A AAG; or
GTA GAC TGC GTA CCA ATT C AAC and GAT GAG TCC TGA CCG A AAT; or
GTA GAC TGC GTA CCA ATT C AAC and GAT GAG TCC TGA CCG A AGT.

Alternatively identification of the markers involves RAPD or genome scanning with microsatellites or SNPs.

In a second aspect the present invention provides a kit for identifying markers for pig semen viability, comprising at least one restriction enzyme and at least one pair of primers.

In one embodiment the kit comprises one or more of the following pairs of primers:

GTA GAC TGC GTA CCA ATT C AAG and GAT GAG TCC TGA CCG A AAC; or
GTA GAC TGC GTA CCA ATT C AAC and GAT GAG TCC TGA CCG A AAG; or
GTA GAC TGC GTA CCA ATT C AAC and GAT GAG TCC TGA CCG A AAT; or
GTA GAC TGC GTA CCA ATT C AAC and GAT GAG TCC TGA CCG A AGT.

The invention will now be described in greater detail with reference to the following example.

### EXAMPLE 1

The aim of this part investigation was to use AFLP to try to identify molecular markers showing an association with semen ‘freezability’ in the boar. The pattern of polymorphisms produced from DNA in the boars with good and poor ‘freezability’ was examined and markers linked to this trait subsequently identified. Reproducibility testing of the AFLP markers identified in boar spermatozoa DNA was also carried out.

Materials and Methods

Experimental Design

129 boars from 3 breeds were assigned to 3 groups based on the post-thaw quality of their spermatozoa. Using a variety of objective tests for sperm quality, 24 boars were classified as good freezers, 63 as medium freezers and 42 boars were bad freezers.

The AFLP technique identifies genetic markers by detecting and evaluating variation in DNA sequence between two phenotypic groups, here good and bad freezers. Any phenotypic variation between sample animals, such as hair colour, will be reflected in the animal's genotype and therefore the AFLP fragment profile. In highly related animals, the only variation between the two sample populations should involve the trait in question. Chance variation between individuals is also seen and this increases as the relatedness between the animals in the study decreases. In this study, we attempted to minimise individual differences by carrying out AFLP analysis on boars from a single breed. Thus we expected that the main difference between the sample boars should be the freezability of their semen.

The Large White population consisted of boars divided between all three freezability groups (13 bad, 25 medium and 9 good freezers), and so was chosen for the AFLP analysis. Animals with medium freezability were not used in the AFLP analysis so the experimental group consisted of 22 animals.

DNA Extraction

Genomic DNA from 22 Large White boars was extracted from spermatozoa and purified according to the following protocol. 100 μl of the sperm rich fraction of raw semen was added to 300 μl extraction buffer A (10 mM Tris-HCl, 5 mM Sucrose, 5 mM MgCl², 1% Triton X-100, adjusted to pH 8.0 using 1M NaOH) and vortexed thoroughly. The diluted semen was centrifuged for 2 min at 7500 rpm and the supernatant discarded. The sperm pellet was then resuspended in 300 μl of extraction buffer B (400 mM Tris-HCl, 60 mM EDTA, 150 mM NaCl, 1% SDS, adjusted to pH 8.0 using 1M NaOH) and incubated for 1 hour at 60° C. 100 μl 5M sodium perchlorate solution was added to the sperm extraction reaction and mixed by inverting 10 times by hand. Pure chloroform (600 μl) was then added and further mixed for 10 min on a rotary mixer. Following centrifugation for 5 min at 7000 g the upper phase was transferred to a new tube and the chloroform step repeated to enhance DNA purification. An equal volume of iso-propanol was added to the second aqueous phase and the samples were mixed by inversion to precipitate the DNA. The DNA pellet was recovered, washed in 70% ethanol and air dried.

Following resuspension of the pellet in 10 mM Tris, 1 mM EDTA, pH 8.0, DNA quality and concentration were assessed on a 1% agarose gel in comparison with known concentrations of phage λ DNA.

The AFLP Technique

The AFLP technique has been previously described in detail (Vos et al., 1995; Vos & Kuiper, 1996; Ajmone et al., 1997). Genomic DNA is digested with two different restriction enzymes, a frequent cutter and a rare cutter. The frequent cutter, TaqI, (4 bp recognition sequence T↓CGA) generates small fragments that amplify efficiently and are in the optimal size range for separation on a denaturing gel.

The rare cutter, EcoRI, (6 bp recognition sequence G↓AATTC) reduces the number of amplified fragments since only the rare cutter-frequent cutter fragments are amplified. Far more DNA fragments are produced by this digestion than could be resolved on a gel so a selective PCR amplification is carried out.

Following digestion, synthetic oligonucleotide adapters are ligated to the restriction fragments to act as primer binding sites. Two selective PCR amplification steps take place, pre-amplification and selective amplification.

Adapter and restriction site sequences (and the single nucleotide adjacent internal to the two restriction sites) are used as targets for primer annealing during the pre-amplification stage. The AFLP pre-amplification primers (also termed +1 primers) are designed to be complementary to the adapter sequence plus the residual restriction site sequence. In addition, primers are synthesised with one extra nucleotide (hence +1) which is chosen by the experimenter; thus there are 4 possible ‘+1’ pre-amplification primers for Eco and for Taq. During pre-amplification, only those restriction fragments which match the +1 selective nucleotides will be amplified. Since the +1 primer is one of 4 possible nucleotide extensions (A,C,T,G) on the EcoRI and TaqI primers, the pre-amplification reduces fragment complexity by 1/16^{th} (EcoRI ¼ and TaqI ¼).

The second selective PCR amplification uses primers with a +3 nucleotide extension. The first position must match that chosen as the +1 extension of the pre-amplification primer but the second and third positions are chosen from all possible combinations. The additional two selective bases on each primer further reduces complexity by 1/256 giving a total reduction (pre-amplification and selective amplification) of 1/4096th. This overall reduction of the restriction fragment complexity produces an optimal number of selectively amplified bands, which could be visualised on an appropriate gel system. By using the different combinations of +3 primers (referred to as primer combinations or PC's), different subsets of restriction fragments can be amplified. The EcoRI primer is labeled with an infrared fluorescent dye (IRD 700 or IRD 800) that can be laser-scanned and which allows the band patterns to be analysed on a LI-COR automated sequencer.

Restriction fragment patterns generated by the selective amplification are a rich source of polymorphisms or AFLP markers. The frequency with which markers are detected depends on the level of sequence polymorphism between the tested DNA samples. Using one primer combination, 50-200 fragments can be analysed on a single gel if resolution is good. Polymorphisms are detected as the presence/absence of a fragment due to: (1) a difference in restriction sites; (2) mutations around the restriction site which match, or are different from, the selective nucleotide extensions; (3) insertions/deletions within the amplified DNA fragment. Each AFLP fragment corresponds to a specific position on the genome and therefore it can be used as a genetic marker if it shows polymorphism, characterised by its size and the primers required for its amplification.

Preparation of AFLP Adapters and Primers

The sequences of EcoRI and TaqI adapters and primers used in the experiment are shown in Table 1.

Adapters were formed by annealing appropriate volumes of the primers. Top and bottom strands were mixed and heated to 95° C. for 5 min, then cooled to room temperature over 15 min. EcoRI adapters were formed by annealing 5 pmole/μl of Eco top strand and 5 pmole/μl of Eco bottom strand. Final concentration is 5 pmole/μl. TaqI adapters were formed by annealing 50 pmole/μl of Taq top strand and 50 pmole/μl of Taq bottom strand. Final concentration was 50 pmole/μl.

Restriction Digests of Genomic DNA

Restriction fragment digestion of extracted spermatozoa DNA was carried out by incubating 250 ng of DNA with 5 units TaqI for 2 hours at 65° C. in 38 μl of restriction/ligation (RL) buffer (50 mM Tris-Acetate pH 7.5; 50 mM MgAc; 250 mM KAc; 25 mM dithiothreitol (DTT); 250 ng/μl bovine serum albumin, BSA). Following this incubation, 2 μl of RL buffer containing 5 units of EcoRI was added and the resulting 40 μl was incubated at 37° C. for 2 hours. Restriction digests were assessed on a 1% agarose gel to ensure the spermatozoa DNA was digested fully.

TABLE 1
uz,7/33 Adapters and primers used in AFLP analysis

| | |
|---|---|
| Name | Sequence |

| | | |
|---|---|---|
| Adapters EcoRI | Eco top strand | CTC GTA GAC TGC GTA CC |
| | Eco bottom strand | AAT TGG TAG GCA GTC TAC |
| Adapters TaqI | Taq top strand | GAC GAT GAG TCC TGA C |
| | Taq bottom strand | CGG TCA GGA CTC AT |
| Primer EcoRI | Pre-amplification | GAC TGC GTA CCA ATT CA |
| Primer TaqI | Pre-amplification | GAT GAG TCC TGA CCG AA |
| Primers EcoRI | E31 | GTA GAC TGC GTA CCA ATT C AAA |
| | E32 | GTA GAC TGC GTA CCA ATT C AAC |
| | E33 | GTA GAC TGC GTA CCA ATT C AAG |
| | E43 | GTA GAC TGC GTA CCA ATT C ATA |
| Primers TaqI | T31 | GAT GAG TCC TGA CCG A AAA |
| | T32 | GAT GAG TCC TGA CCG A AAC |
| | T33 | GAT GAG TCC TGA CCG A AAG |
| | T34 | GAT GAG TCC TGA CCG A AAT |
| | T38 | GAT GAG TCC TGA CCG A ACT |
| | T39 | GAT GAG TCC TGA CCG A AGA |
| | T42 | GAT GAG TCC TGA CCG A AGT |

Selective nucleotides are shown in bold text.

Ligation of Adapters

Digested DNA fragments have staggered ends for the annealing of AFLP adapters. The adapters consist of a core sequence and a restriction enzyme specific sequence. To ligate adapters, 10 μl of RL buffer containing 5 pmole EcoRI adapters, 50 pmole TaqI adapters, 1 Weiss unit T4 DNA ligase (1 Weiss unit is approximately 60 cohesive end units) and 1 μl of 10 mM ATP was added to the restriction digest reaction. This 50 μl restriction/ligation reaction was incubated at 37° C. overnight. Ligated template DNA was diluted 1:10 with 10 mM Tris-HCl, 0.1 mM EDTA (pH 8.0) and stored at −20° C. before pre-amplification.

Pre-Amplification of Template DNA

AFLP pre-amplification primers were composed of a core sequence, enzyme-specific sequence and a single nucleotide extension (+A; Table 1). Five microlitres of diluted ligated template DNA was added to a PCR reaction mix containing: 5 μl 10×PCR buffer (200 mM Tris-HCl, pH 8.4; 500 mM KCl), 3 μl MgCl₂ (25 mM), 5 μl dNTP's (2 mM), 0.2 μl AmpliTaq polymerase and 50 ng each of the EcoRI and TaqI pre-amplification primers carrying one selective nucleotide in a total volume of 50 μl.

The PCR pre-amplification consisted of 20 cycles with a profile of 30 sec at 94° C. to denature the DNA, 1 min at 56° C. for primer annealing and 1 min at 72° C. for primer extension, followed by 5 min at 72° C. for the completion of partial amplifications. PCR amplification was carried out using the Gene Amp PCR system 9700 (Applied Biosystems). The preamplified template was diluted 20-fold with 10 mM Tris-HCl, 0.1 mM EDTA (pH 8.0) before the selective PCR amplification.

Selective Amplification

Five microlitres of diluted preamplified template DNA was added to a 15 μl PCR reaction mix containing; 2 μl 10×PCR buffer (200 mM Tris-HCl, pH 8.4; 500 mM KCl), 1.2 μl MgCl₂ (25 mM), 2 μl dNTP's (2 mM) and 0.1 μl AmpliTaq polymerase. PCR primers carrying three arbitrarily chosen selective nucleotides (Table 1) were used in this amplification. 15 ng EcoRI primer terminally labeled with infra-red dye (IRD) and 30 ng unlabelled TaqI primer were added. Either of two IRD's, fluorescing at wavelengths of either 700 nm or 800 nm, were used to label the EcoRI primers allowing two primer combinations to be run on a gel simultaneously. The LI-COR 4200 sequencer incorporates two separate sets of optically tuned lasers and detectors which can distinguish between the fluorescence signals from restriction fragments labeled with IRD 700 and IRD 800, producing two independent AFLP profiles from a single gel run.

Amplification was performed for 13 cycles with the following profile: 30 sec at 94° C. for DNA denaturation, 30 sec at 65° C. as an annealing step and 2 min at 72° C. for primer extension. In each cycle, the annealing temperature was reduced by 0.7° C. down to 56° C. This ‘touch down’ reduction in annealing temperature promotes high-stringency amplification. Immediately following these 13 cycles, a further set of 23 cycles was performed for 30 sec at 94° C., 30 sec 56° C. and 2 min at 72° C. The PCR reaction was then cooled to a holding temperature of 6° C. PCR amplification was carried out using the Gene Amp PCR system 9700.

Detection and Scoring of AFLP Markers

Two microlitres of labeled amplified DNA were mixed with 2 μl of microSTOP loading buffer (96% formamide, 20 mM EDTA, red and blue tracking dye) and denatured by heating to 90° C. for 2 min and snap cooling on ice. Two microlitres of this mix were loaded onto a 6% polyacrylamide denaturing gel.

Two microlitres of SequaMark 10 bp ladder DNA size markers labeled with either IRD 700 or IRD800 and prepared according to the manufacturer's instructions were also loaded onto the gel. Size markers serve as a reference point for scoring AFLP restriction fragments and a general control for the resolving power of each gel. SequaMark produces a DNA ladder with bands every 10 bases up to 500 bp.

DNA fragments were separated on a 6% denaturing polyacrylamide sequencing gel. The gel was prepared using 6% acrylamide, 0.25% methylene bisacryl, 1×TBE (89 mM Tris-HCl, 89 mM Boric acid, 2 mM EDTA) and 6M urea, pH 8.3 (‘Sequagel 6’). To 35 ml of gel solution, 280 μl of 10% ammonium persulphate were added and gels were cast using a LI-COR 41 cm gel assembly.

TBE (100 mM Tris-HCl, 100 mM Boric acid, 2 mM EDTA) was used as a running buffer. Gels were run at constant watts (40 W) for 5 hours.

LI-COR Automated Sequencer

Gel electrophoresis was performed on a LI-COR automated sequencer. The AFLP fragment image data were collected and the image cropped using BaseImagIR software (version 4.0). The efficiency and repeatability of the LI-COR automated DNA sequencer and associated BaseImagIR software has been previously validated (Qui et al., 1999).

The LI-COR sequencer utilises an extremely sensitive infrared detection system. Two sets of optically tuned lasers measure fluorescence signals from independent infrared fluorescent dyes (IRD 700 and IRD 800), each labeling one of two distinct AFLP profiles, running in parallel, on the same gel. The two-channel detection system avoids creating errors in profiles due to fluorescence overlap by using IRD's which are separated by 100 nm.

Gel images, similar to autoradiograms, for each AFLP profile are collected in real time and displayed in BaseImagIR analysis software. These gel profiles are then imported into AFLP-Quantar image analysis software (KeyGene) to identify the presence of polymorphisms.

Image Analysis Software

The AFLP-Quantar image analysis package (hereafter referred to as Quantar) is a specialised software application for the analysis of AFLP DNA fingerprints. Quantar is based on the Windows platform and can be used to analyse gels from fluorescent, radioactive and infrared detection systems. Quantar was used to identify and measure specific AFLP band patterns in a gel pixel image produced by the LI-COR sequencer and exported from the BaseImagIR software.

The semi-automated Quantar software is used to score polymorphic marker bands identified in the AFLP profile on a dominant basis (presence/absence of a band); it does not score markers co-dominantly. (present high intensity/present low intensity/absent band). Quantar uses information from ‘constant’ bands (ie. monomorphic bands, present in every sample/lane) such as position, shape, intensity and relative mobility, to correct for variations in gel running and image artefacts, ensuring that marker classification is precise and reliable. Profiles were scored using the set software parameters, but band classifications were confirmed by the operator to ensure that no markers were misclassified. The band pattern of SequaMark DNA size markers was programmed into the Quantar software and evaluated on each sequence gel, to allow accurate automated sizing of polymorphic bands.

Statistical Analysis

Summary statistics for AFLP gel profiles from each boar, DNA extraction and primer combination, were calculated. Logistic regression (Altman, 1991) was used to relate the dichotomous presence/absence of an AFLP marker band with classifications of semen freezability (good/bad) and individual assessments of semen quality after cryopreservation. Statistical analyses were performed using Statistica for Windows, version 4.5.

Marker Reliability

The reproducibility of the AFLP technique was tested across independent DNA extractions and at both the pre-amplification and selective amplification stages to check the PCR technique produced a consistent DNA template. Three AFLP band profiles were compared for each stage of the protocol.

Results

Marker Reliability

Independent DNA extractions and template preparations gave rise to identical AFLP profiles. The AFLP protocol proved to be highly reproducible with bands consistently identifiable between boars, gels and primer combinations.

In this study fragment profiles were investigated using Quantar dominant marker scoring. Although the fluorescence levels of amplified fragments were comparable between DNA samples, the variable operator skill when loading gels resulted in a small variation in the volume of DNA loaded onto each gel. Despite this minimal variation, the resulting difference in DNA volume between lanes prevented the use of co-dominant scoring, as fluorescence levels were not directly comparable.

Automated scoring of the gel profiles by the Quantar package proved to be fast (each gel evaluated in <30 min), accurate and reproducible. Any questionable band pattern identified by the package could be screened by the operator and if necessary removed from the analysis, thus preventing inaccurate scoring.

Identification of Potential Markers of Semen ‘Freezability’

Twenty-eight primer combinations generated 2182 bands of which 421 were polymorphic (where some animals were positive for an amplified restriction fragment and others were negative). The average number of polymorphic amplified fragments per primer combination was 15 with a range between 4 and 31 bands.

Distinct differences in AFLP profiles were observed between boars classified as ‘good’ and ‘poor’ freezers. Good is defined as above 50% motility post-thaw, average as 20-50% and poor is less than 20% motility. Each amplified fragment was examined between good and bad freezers. The number of times a specific fragment was amplified in each group of good and bad was calculated and related to semen freezability. AFLP markers were identified by logistic regression analyses correlating the presence/absence of an amplified fragment with the classification of good/bad freezability (Table 2). A marker was identified when one group of boars had statistically more amplified fragments than the other group of boars.

Logistic regression was also used to identify significant relationships between the AFLP freezability markers and individual sperm quality assessment tests (Table 2). Due to the large amount of data analysed, only significant correlations are included in Table 2. All absent regression data can be assumed to be non-significant.

AFLP marker 11 was significantly related to the total percentage motile spermatozoa and the % of progressively motile spermatozoa following cryopreservation; markers 11, 7, 9 and 8 were related to the percentage of spermatozoa with intact plasma membranes (SYBR-14 positive); markers 11, 7 and 9 related to type 3 morphology; and markers 8 and 1 were significantly related to type 1 morphology (Table 2).

The number of AFLP markers for semen freezability at significance levels of P<0.01 and P<0.005, generated by different primer combinations, are shown in Table 3. Markers are described by both fragment size (number of base pairs) and primer combination (Table 4) for application in future screening programmes.

TABLE 2
Logistic regression analysis of the presence/absence of AFLP markers
with classifications of good/bad freezability and semen quality
assessments.

| | | | | Significance | Significance of |
|---|---|---|---|---|---|
| Mar- | | Regression | | of variables | logistic model |
| ker | Variable | coefficient | SE | (P) | (P) |
| 1 | G/B | 0.599 | 1 | 0.005 | 0.005 |
| 1 | % Gp.1 | 0.458 | 0.07 | 0.03 | |
| 2 | G/B | 0.643 | 1 | 0.005 | 0.003 |
| 3 | G/B | 0.650 | 1 | 0.005 | 0.004 |
| 4 | G/B | 0.740 | 1 | 0.001 | 0.0007 |
| 5 | G/B | 0.589 | 1 | 0.005 | 0.005 |
| 6 | G/B | 0.759 | 1 | 0.001 | 0.0001 |
| 7 | G/B | 0.639 | 1 | 0.005 | 0.005 |
| 7 | % SYBR14 | 0.775 | 0.1 | 0.0009 | |
| 7 | % Gp.3 | 0.978 | 0.06 | 0.0001 | |
| 8 | G/B | 0.639 | 1 | 0.005 | 0.005 |
| 8 | SYBR14 | 0.436 | 0.1 | 0.04 | |
| 8 | % Gp.1 | 0.978 | 0.06 | 0.0001 | |
| 9 | G/B | 0.641 | 1 | 0.005 | 0.005 |
| 9 | % SYBR14 | 0.752 | 0.07 | 0.001 | |
| 9 | % Gp.3 | 0.842 | 0.05 | 0.0006 | |
| 10 | G/B | 0.663 | 1 | 0.005 | 0.003 |
| 11 | G/B | 0.662 | 1 | 0.004 | 0.004 |
| 11 | % motile | 0.503 | 0.12 | 0.02 | |
| 11 | % prog.mot | 0.436 | 0.41 | 0.04 | |
| 11 | % SYBR14 | 0.639 | 0.06 | 0.005 | |
| 11 | % Gp.3 | 0.413 | 0.07 | 0.05 | |
| 12 | G/B | 0.600 | 1 | 0.003 | 0.005 |
| 13 | G/B | 0.662 | 1 | 0.006 | 0.005 |
| 14 | G/B | 0.797 | 1 | 0.0008 | 0.005 |
| 15 | G/B | 0.727 | 1 | 0.001 | 0.004 |
| 16 | G/B | 0.752 | 1 | 0.001 | 0.0008 |
| G/B are classifications of good and bad freezers, % SYBR14 refers to the percentage of spermatozoa with intact plasma membranes in the thawed ejaculate, % Gp.1/3 refers to the | | | | | |
| # percentage of spermatozoa in the ejaculate with either sub-population 1 or sub-population 3 morphology before freezing. | | | | | |

TABLE 3
Identification of polymorphisms which are significantly
correlated with boar semen ‘freezability’
(P < 0.01 and P < 0.005)

| Primer | No. | No. polymorphisms | No. polymorphisms |
|---|---|---|---|
| combination | Polymorphisms | P < 0.01 | P < 0.005 |
| E31/T31 | 8 | 0 | 0 |
| E31/T32 | 20 | 1 | 1 |
| E31/T33 | 14 | 1 | 0 |
| E31/T34 | 8 | 3 | 0 |
| E31/T38 | 18 | 0 | 0 |
| E31/T39 | 20 | 1 | 1 |
| E31/T42 | 14 | 2 | 2 |
| E32/T31 | 6 | 2 | 2 |
| E32/T32 | 31 | 1 | 0 |
| E32/T33 | 23 | 2 | 1 |
| E32/T34 | 22 | 2 | 1 |
| E32/T38 | 22 | 2 | 0 |
| E32/T39 | 12 | 0 | 0 |
| E32/T42 | 12 | 3 | 2 |
| E33/T31 | 19 | 1 | 0 |
| E33/T32 | 15 | 3 | 1 |
| E33/T33 | 25 | 1 | 1 |
| E33/T34 | 16 | 0 | 0 |
| E33/T38 | 10 | 2 | 1 |
| E33/T39 | 7 | 0 | 0 |
| E33/T42 | 8 | 0 | 0 |
| E43/T31 | 18 | 0 | 0 |
| E43/T32 | 12 | 1 | 1 |
| E43/T33 | 20 | 0 | 0 |
| E43/T34 | 11 | 0 | 0 |
| E43/T38 | 14 | 0 | 0 |
| E43/T39 | 4 | 0 | 0 |
| E43/T42 | 12 | 2 | 2 |
| Total | 421 | 30 | 16 |

TABLE 4
Size (base pairs) of each AFLP freezability marker
(logistic regression P < 0.005)

| Freezability marker | Fragment size (base pairs) | Primer combination |
|---|---|---|
| 1 | 411 | E31 T32 |
| 2 | 372 | E31 T39 |
| 3 | 502 | E31 T42 |
| 4 | 106 | E31 T42 |
| 5 | 460 | E32 T31 |
| 6 | 220 | E32 T31 |
| 7 | 481 | E32 T33 |
| 8 | 286 | E32 T34 |
| 9 | 438 | E32 T42 |
| 10 | 371 | E32 T42 |
| 11 | 258 | E33 T32 |
| 12 | 467 | E33 T33 |
| 13 | 311 | E33 T38 |
| 14 | 233 | E43 T32 |
| 15 | 431 | E43 T42 |
| 16 | 161 | E43 T42 |

Discussion

It was possible to identify regions of the genome correlated with variation in semen freezability and to generate markers associated with the genes related to this complex trait. The DNA markers identified using AFLP are expected to be useful in the selection of boars for use in projects involving semen cryopreservation.

DNA Markers and Sperm Quality Assessment

Potential markers for semen freezability were identified by exploring relationships between the presence/absence of an amplified DNA fragment and classifications of good and bad freezers. These classifications were determined by incorporating all sperm quality assessment data in a multivariate PATN analysis and objectively identifying groups of animals based on the ability to survive the freeze-thaw process. It is important that the method of classifying a boar as a good or bad freezer is accurate. If the assessment of semen freezability and subsequent classification of animals is inadequate, differences in the DNA profiles will not be discrete, preventing the identification of informative markers. Utilising a large number of sperm quality assessment techniques provided a comprehensive assessment of semen quality following cryopreservation, and therefore improved the chances of identifying markers linked to genes controlling freezability.

It is important to note that although DNA markers linked to semen freezability were identified, there were also relationships between the DNA profile and certain individual assessments of sperm quality after thawing. No specific viability test correlated with all DNA markers, indicating that semen freezability cannot be assessed by a single technique such as motility or acrosome integrity. Rather semen freezability must be defined using a combination of all sperm quality assessments. This limited relationship between the DNA profile and specific viability tests indicates that the genes responsible for good freezability may not act directly on a particular sperm function, but have a more subtle influence on the response of spermatozoa to the freeze-thaw process. Alternatively, an array of markers linked to different genes may all influence different biophysical aspects of the cellular freezing response. This would explain why different markers are related to different methods of viability testing.

Summary

We have demonstrated the feasibility of generating DNA markers associated with complex traits. We were able to identify repeatable polymorphic markers between two populations of Large White boars classified on the basis of their semen ‘freezability’. Identified marker associations with semen ‘freezability’ can be used in marker assisted selection programmes to help address the problem of ‘poor freezers’ in pigs. Freezability markers may also help to address semen preservation problems in other mammalian species, including livestock, domestic and endangered species.

1. A method for identifying the presence or absence of genetic markers associated with post-thaw viability of cryopreserved pig semen comprising the steps of:
(a) Obtaining nucleic acid from a pig; and
(b) Identifying the presence or absence of one or more markers for semen viability.

2. A method as claimed in claim 1 wherein the nucleic acid is obtained from a pig prior to sexual maturity.

3. A method as claimed in claim 1 wherein the nucleic acid is obtained from pig semen.

4. A method as claimed in any of claims 1 to 3 wherein the nucleic acid is DNA.

5. A method as claimed in any of claims 1 to 4 wherein identification of the markers involves amplified restriction length polymorphism technology (AFLP).

6. A method as claimed in claim 5 wherein the restriction enzymes produce fragments of around 300 base pairs to 500 base pairs.

7. A method as claimed in claim 5 or claim 6 wherein the AFLP utilises the restriction enzymes EcoRI and TaqI.

8. A method as claimed in any one of claims 5 to 7 wherein the identification involves amplification using one or more of the following pairs of primers:
GTA GAC TGC GTA CCA ATT C AAG and GAT GAG TCC TGA CCG A AAC; or
GTA GAC TGC GTA CCA ATT C AAC and GAT GAG TCC TGA CCG A AAG; or
GTA GAC TGC GTA CCA ATT C AAC and GAT GAG TCC TGA CCG A AAT; or
GTA GAC TGC GTA CCA ATT C AAC and GAT GAG TCC TGA CCG A AGT.

9. A method according to any of claims 5 to 8 wherein the marker is one or more of the markers listed in Table 4.

10. A method as claimed in any of claims 1 to 4 wherein identification of the markers involves random amplified polymorphic DNA analysis (RAPD).

11. A method as claimed in any of claims 1 to 4 wherein identification of the markers involves genome scanning with microsatellites or single nucleotide polymorphisms (SNPs).

12. A kit for identifying markers for pig semen viability, comprising at least one restriction enzyme and at least one pair of primers.

13. A kit as claimed in claim 12 wherein the kit comprises one or more of the following pairs of primers:
GTA GAC TGC GTA CCA ATT C AAG and GAT GAG TCC TGA CCG A AAC; or
GTA GAC TGC GTA CCA ATT C AAC and GAT GAG TCC TGA CCG A AAG; or
GTA GAC TGC GTA CCA ATT C AAC and GAT GAG TCC TGA CCG A AAT; or
GTA GAC TGC GTA CCA ATT C AAC and GAT GAG TCC TGA CCG A AGT.
